(19) United States
(12) Patent Application Publication
(10) Pub. No.: US 2026/0027311 A1
(43) Pub. Date: Jan. 29, 2026

(54) A POSITIVE AIRWAY PRESSURE DEVICE WITH A NOISE-REDUCING AIR PASSAGE

(71) Applicant: WALLENBERG UNION LLC, NEWARK, DE (US)

(72) Inventor: David LUO, NEWARK, DE (US)

(73) Assignee: WALLENBERG UNION LLC, NEWARK, DE (US)

(21) Appl. No.: 18/784,204

(22) Filed: Jul. 25, 2024

(51) Int. Cl. A61M 16/00

(52) U.S. Cl. CPC A61M 16/0066; A61M 16/0003; A61M 2202/0007; A61M 2202/02; A61M 2205/0216; A61M 2205/0222; A61M 2205/025; A61M 2205/103; A61M 2205/3331; A61M 2205/42

(57) ABSTRACT

A device to provide positive airway pressure for use in pressure therapy to treat respiratory-related diseases. The device includes a casing configured to enclose and protect its internal components from external environmental interference. Inside the casing, a noise-reducing air passage is provided, which has an inlet, an outlet, an inner wall, and an outer wall, configured to receive external gas and allow pressurized gas to flow out. The noise-reducing air passage also houses a blower assembly, a core component of the device, configured to pressurize the breathable gas entering the noise-reducing air passage and then output it. Specifically, the blower assembly includes an impeller, a motor, and its internal gas channel. The blower assembly also has an intake port and an exhaust port.

### TECHNICAL FIELD

The present disclosure pertains to a device to provide positive airway pressure for use in pressure therapy to treat respiratory-related diseases. The device includes a casing, a noise-reducing air passage, and a blower assembly provided inside the noise-reducing air passage.

### BACKGROUND

Sleep plays a crucial role in promoting physical and mental health. Issues related to sleep quality, duration, and frequency can severely disrupt normal physical, psychological, social, and emotional functions. These issues are mainly caused by sleep disorders, which affect millions of people worldwide. In the United States, millions of people suffer from chronic sleep or wakefulness disorders such as narcolepsy, insomnia, restless leg syndrome, and sleep apnea. Among these conditions, narcolepsy is characterized by excessive daytime sleepiness and brief involuntary sleep episodes, which can interfere with daily activities and social interactions. Additionally, around 70% of affected individuals experience sudden muscle weakness attacks, known as cataplexy. Sleep disorders can occur at any age, particularly in middle-aged men, and are extremely harmful to people's physical and mental health. Sleep disorders can lead to various complications, including depression, bipolar disorder, and schizophrenia. Snoring is often a primary indicator of these symptoms. Given the detrimental effects of sleep disorders, diagnosing these conditions is essential for timely protection of physical and mental health.

People breathe through their nose and mouth, but during sleep, various factors can narrow the patient's airway. When breathing through this narrowed passage, snoring occurs. When the cause of the narrowed airway is in the nose, it is called “nasal snoring,” and when the cause is in the tongue or tonsils, it is referred to as “throat snoring.” The primary cause of nasal snoring is nasal congestion due to colds or allergic rhinitis, which causes inflammation of the nasal mucosa and narrows the nasal passages, producing sound. Some individuals have a deviated nasal septum, where the bone in the center of the nose is crooked, also contributing to snoring. On the other hand, throat snoring mainly occurs when the tongue slides back and blocks the airway during sleep. Enlarged or inflamed tonsils can also cause snoring. In most cases, nasal snoring can be cured by treating the inflammation causing the congestion, and if necessary, correcting the deviated septum. However, throat snoring requires more attention as it can lead to sleep apnea, where breathing repeatedly stops during sleep. This condition generally requires intervention. Additionally, some people snore with their mouth closed, indicating a potential risk for sleep apnea and other serious health conditions. Overall, the causes of respiratory-related diseases are varied, including physical constitution, fatigue, and sudden health conditions that temporarily narrow the airway. In most cases, once the underlying issue is resolved, the symptoms of respiratory diseases subside. Results from experiments and data analysis indicate that, for most adults, including the elderly, airway treatment can reduce the risk of respiratory events during sleep, decrease daytime sleepiness, lower the risk of motor vehicle accidents, and improve systemic blood pressure, symptoms of gastroesophageal reflux disease, blood sugar control in diabetic patients, and quality of life.

Therefore, by providing a steady airflow to the airway and keeping it open, the device provided by this disclosure prevents the collapse and obstruction of the airway caused by muscle relaxation during inhalation, especially when sleeping in a supine position. This method not only addresses snoring issues but also mitigates health risks associated with obstructive sleep apnea. Pressure therapy for respiratory-related symptoms can improve sleep quality and reduce health problems such as cardiovascular diseases and daytime fatigue. Recognizing and adopting effective pressure therapy is essential for improving respiratory-related conditions and overall quality of life.

### SUMMARY

The objective of this disclosure is to provide a novel device to deliver positive airway pressure, optimized in terms of structure and component coordination, to achieve better performance on the existing basis. This optimization facilitates the manufacturing of the device and its rapid adaptation to the market. The device utilizes an efficient structure to reduce noise within the smallest possible size, overcoming the limitations of existing technologies. It offers a more effective solution with broader application implementations for safely delivering continuous positive airway pressure to the patient's airway for the treatment of sleep-related breathing disorders.

In one embodiment, a device to provide positive airway pressure is provided. The device includes a casing configured to enclose internal components; a noise-reducing air passage that includes an inlet, an outlet, an inner wall, and an outer wall, where a central axis of the inlet and a central axis of the outlet form an angle; and a blower assembly that includes an intake port and an exhaust port, with a central axis of the intake port and a central axis of the exhaust port being perpendicular to each other. The breathable gas enters the intake port and flows to the exhaust port tangentially to a rotation of an impeller. The blower assembly includes a blower housing, the impeller, a motor, and an internal gas channel. At least part of the internal gas channel of the blower assembly is located below the impeller. The volume ratio of the blower assembly to the noise-reducing air passage is between 3 to 18, and a distance between the blower assembly and the inner wall of the noise-reducing air passage is greater than or equal to 3 mm when the blower assembly is secured within the noise-reducing air passage.

In one embodiment, in a top view, the intake port of the blower assembly is provided at an approximately central position of the noise-reducing air passage.

In one embodiment, the noise-reducing air passage includes a first chamber, a second chamber, and a wall to separate the first chamber from the second chamber, and an opening is provided on the wall.

In one embodiment, the inlet and the outlet of the noise-reducing air passage are not on the same horizontal plane.

In one embodiment, an area of the outlet of the noise-reducing air passage is 0.7 to 1.5 times an area of the exhaust port of the blower assembly.

In one embodiment, the device further includes a bracket, and the blower assembly is secured within the noise-reducing air passage by the bracket.

In another embodiment, a device to provide positive airway pressure is provided, configured to deliver breathable gas into a patient's airway for treatment of respiratory-related diseases. The device includes a casing configured to enclose internal components; a noise-reducing air passage that includes an inlet, an outlet, an inner wall, and an outer wall, and is configured to transmit the breathable gas; and a blower assembly that includes an intake port and an exhaust port, where the blower assembly is configured to pressurize the breathable gas that enters the noise-reducing air passage. The blower assembly includes a blower housing, an impeller, a motor, and an internal gas channel, with at least part of the internal gas channel located below the impeller. The device further includes a bracket, and the blower assembly is secured to the noise-reducing air passage at an approximately central position in a vertical direction by the bracket. The intake port of the blower assembly and the inlet of the noise-reducing air passage are non-concentric, and the exhaust port of the blower assembly and the outlet of the noise-reducing air passage are concentric. And a height of the noise-reducing air passage differs from a height of the blower assembly by at least 5 mm.

In one embodiment, the blower assembly is provided within the noise-reducing air passage, and an axis of the intake port of the blower assembly is parallel to a horizontal plane.

In one embodiment, an outlet pipe is provided at the outlet of the noise-reducing air passage.

In one embodiment, the exhaust port of the blower assembly is sealingly connectable to the outlet of the noise-reducing air passage by an elastomer.

In one embodiment, the bracket has at least two different wall thicknesses.

In one embodiment, a contact area between the bracket and the blower assembly is at least 220 mm².

In one embodiment, at least part of the bracket is in contact with the inner wall of the noise-reducing air passage.

In yet another embodiment, a device to provide positive airway pressure is provided, configured to deliver breathable gas into a patient's airway for treatment of respiratory-related diseases. The device includes a casing configured to enclose internal components; a noise-reducing air passage that includes an inlet, an outlet, an inner wall, and an outer wall, and is configured to transmit the breathable gas; and a blower assembly provided within the noise-reducing air passage that includes an intake port and an exhaust port, where the blower assembly is configured to pressurize the breathable gas that enters the noise-reducing air passage. The exhaust port of the blower assembly is sealingly connectable to the outlet of the noise-reducing air passage by an elastomer. The blower assembly includes a blower housing, an impeller, a motor, and an internal gas channel, with at least part of the internal gas channel located below the impeller. The impeller is a closed impeller.

In one embodiment, the impeller has a central opening, and the intake port of the blower assembly is greater than or equal to the central opening of the impeller.

In one embodiment, the blower assembly is provided within the noise-reducing air passage, and an axis of the intake port of the blower assembly is perpendicular to a horizontal plane.

In one embodiment, the inlet and the outlet of the noise-reducing air passage are not on the same horizontal plane.

In one embodiment, the inner wall and the outer wall of the noise-reducing air passage include one of the following materials: polypropylene, polycarbonate, polyethylene terephthalate-1,4-cyclohexane dimethanol ester, polyamide, or polyetheretherketone.

In a further embodiment, a device to provide positive airway pressure is provided, configured to deliver breathable gas into a patient's airway for treatment of respiratory-related diseases. The device includes a casing configured to enclose internal components; a noise-reducing air passage that includes an inlet, an outlet, an inner wall, and an outer wall, and is configured to transmit the breathable gas; and a blower assembly that includes an intake port and an exhaust port, where the blower assembly is configured to pressurize the breathable gas that enters the noise-reducing air passage. The blower assembly is secured inside the noise-reducing air passage, with the intake port of the blower assembly and the inlet of the noise-reducing air passage being non-concentric, and the exhaust port of the blower assembly and the outlet of the noise-reducing air passage being concentric. The blower assembly includes a blower housing, an impeller, a motor, and an internal gas channel, with at least part of the internal gas channel located below the impeller. The impeller is a closed impeller, and the breathable gas enters through the intake port of the blower assembly and flows to the exhaust port tangentially to a rotation of the impeller.

In one embodiment, the noise-reducing air passage includes a first chamber, a second chamber, and a wall to separate the first chamber from the second chamber, with an opening provided on the wall.

In one embodiment, an axis of the inlet of the noise-reducing air passage is parallel or perpendicular to an axis of the intake port of the blower assembly.

In one embodiment, the exhaust port of the blower assembly is sealingly connectable to the outlet of the noise-reducing air passage by an elastomer.

In one embodiment, the impeller has a central opening, and the intake port of the blower assembly is greater than or equal to the central opening of the impeller.

The implementation of a device to provide positive airway pressure provided by this disclosure at least has the following benefits:
1. By applying scientific theories and experimental data, the internal structure and the placement of components (such as the blower assembly) are optimized to achieve a more efficient and noise-reducing configuration. (1) During the development and design of the internal noise-reducing air passage, the proportional relationship between the blower assembly and the noise-reducing air passage was thoroughly explored to understand its impact on the noise levels of the device. Through extensive data review, designing various proportioned volumes of blower assemblies and noise-reducing air passages, and conducting tests in standard soundproof rooms to ensure accuracy and reliability of the results, it was concluded that a volume ratio of 3 to 18 between the blower assembly and the noise-reducing air passage achieves a more notable noise reduction effect. Utilizing precise theoretical data, the volume ratio between the noise-reducing air passage and the blower assembly was specified more accurately, resulting in better noise reduction performance. Given the increasing demand for quiet performance of the devices to provide positive airway pressure, this disclosure provides an innovative solution to meet this need. By standardizing data, it offers a safe and effective noise reduction method, providing patients with a quieter device, aligning with the growing expectations and requirements for silent therapeutic devices. (2) Positioning the blower assembly centrally within the noise-reducing air passage facilitates a more uniform airflow route and extends the airflow path. In a top view, the blower assembly is provided in a approximately central position within the noise-reducing air passage and the intake port of the blower assembly is also provided at an approximately central position of the noise-reducing air passage. This central placement of the intake port ensures that air can enter the blower assembly evenly from all directions, avoiding localized excessive or insufficient air flows. This configuration reduces the variation in airflow velocity and enhances the stability and uniformity of the airflow. This method also disperses and balances the airflow before it enters the blower assembly, reducing the potential for airflow vibrations and further diminishing turbulence and noise produced by airflow twists and turns. Having the blower assembly provided in a vertically central position within the noise-reducing air passage reduces the distance between the blower assembly and the inner wall of the noise-reducing air passage, which helps to reduce the airflow resistance and pressure fluctuations, thereby enhancing the efficiency and stability of the noise-reducing air passage. The placement of the blower assembly in a vertically central position also extends the airflow path, adding a vertical dimension to the horizontal flow path, which helps the airflow to move more uniformly and stably, thus reducing noise. (3) Setting the outlet and inlet of the noise-reducing air passage on two different planes and with their axes at a perpendicular angle reduces the potential for noise accumulation. When the airflow enters the noise-reducing air passage through the inlet, the characteristics and velocity distribution of the fluid often make the inlet the primary source of airflow noise. Thus, configuring the inlet and outlet perpendicularly helps to separate these two areas, positioning the major noise source away from the patient to achieve noise reduction. (4) Utilizing a quieter centrifugal blower assembly is an effective method to reduce the noise of the device. Since the blower assembly is the principal source of noise in the device, using a quieter blower instead of adding more noise-reduction structures or foam materials fundamentally lowers the noise level of the device. This approach is not only more effective but also more convenient and can enhance the noise performance of the device, improving user comfort and experience quality. In this disclosure, a centrifugal blower assembly is used, which not only produces less noise compared to axial-flow blower assemblies but also offers higher efficiency. Once the blower assembly type is selected, the noise-reducing air passage of this disclosure is designed to complement the chosen blower assembly to achieve the best possible noise reduction in this configuration.
2. Lowering the internal gas channel within the blower assembly presents multiple advantages over existing blower designs in terms of noise reduction and operational efficiency. (1) By relocating the internal gas channel to an approximately central position within the blower assembly, the incoming air passes through the motor, thereby removing heat generated by the motor during operation. This aids in cooling the motor and maintaining it within an optimal temperature range, preventing overheating issues associated with prolonged use. This adjustment not only enhances the motor's efficiency and lifespan but also improves the overall efficiency and durability of the blower assembly. (2) In conventional blower assemblies on the market, the gas channel is designed at the impeller, where the air, immediately after being pressurized by the impeller, enters the gas channel and is expelled. This setup results in sudden and rapid changes in airflow at the impeller and gas channel, which can cause turbulence or irregular flows, thereby increasing the noise level of the blower assembly. Moreover, these sudden changes can induce airflow oscillations, further exacerbating noise issues. In contrast, sinking the gas channel to a more central position in the blower assembly provides a buffer zone for the pressurized air, allowing the airflow to move more smoothly and uniformly along the path of the gas channel, thereby reducing internal turbulence and irregular flows, and consequently, decreasing noise. (3) When employing a closed impeller in the blower assembly, unlike an open impeller, the blades are enclosed within a sealed casing with a central opening. Air enters through this central opening and moves through the channels formed by the impeller blades, enabling the airflow to exit at a higher pressure from the more enclosed structure. Open impellers, having a more open structural design, allow air to flow freely on both sides of the impeller without much directional constraint. Therefore, using a closed impeller necessitates lowering the gas channel within the blower assembly to provide additional space for integrating and guiding the pressurized airflow, thus enhancing the performance and efficiency of the blower assembly. (4) Designing the gas channel at the central position within the blower assembly also stabilizes the assembly within the noise-reducing air passage. Traditional blower assemblies available on the market often position the internal gas channel at the top of the unit, whereas this disclosure lowers it, effectively shifting the center of gravity downward towards the center of the blower assembly. This design reduces mechanical vibrations caused by changes in airflow pressure and velocity, enhancing the operational stability and safety of the blower. Additionally, it aids the support bracket in securing the blower assembly more stably within the noise-reducing air passage, thereby reducing some motor noise. (5) The disclosure primarily utilizes a centrifugal blower assembly placed inversely within the noise-reducing air passage, altering the direction and speed of air flow. This orientation reduces the formation of vortices and eddies as air flows through the impeller, directed in a specific orientation. Along with the special chamber structure in the disclosure, the noise at the exhaust port of the blower assembly is contained, thereby reducing noise production. In this disclosure, the overall volume of the blower assembly and the volume of the noise-reducing air passage are calculated, and by configuring the gas channel and the components within the chambers (such as the bracket), sound energy is converted into kinetic energy, thereby reducing noise. The noise-reducing air passage is set to enclose the blower assembly at the center and includes chambers at the blower assembly's intake port to use the air or structures within the chambers to lower noise levels. The cooperation between the gas channel and the noise-reducing air passage further adjusts the airflow path to create a vertical disparity, which increases the path of airflow while maintaining the device's flow rate and reduces turbulence at the junctions of various components and chambers, resulting in a smoother airflow path.
3. The bracket system in this disclosure, compared to existing suspensions on the market, not only assists in noise reduction but also provides greater stability for the blower assembly. Part of the bracket is configured to connect to the bottom wall of the chamber housing the blower, with elastomers at the top and around the exhaust port of the blower assembly forming integral parts of the bracket. This design secures the blower assembly at several fixed points, firmly anchoring it within one of the chambers of the noise-reducing air passage. This is more stable than conventional suspension systems and reduces the risk of damage to the blower assembly from vibrations or external impacts. Moreover, the bracket encloses the intake port and exhaust port of the blower assembly, supporting the assembly while also sealing the ports to prevent air leakage.

### BRIEF DESCRIPTION OF THE DRAWINGS

FIG. 1 is a three-dimensional schematic diagram of a device to provide positive airway pressure in accordance with one embodiment;

FIG. 2 is an exploded structural view of a device to provide positive airway pressure in accordance with one embodiment;

FIG. 3 is a three-dimensional schematic diagram of the noise-reducing air passage of a device to provide positive airway pressure in accordance with one embodiment;

FIG. 4 is a cross-sectional view of the noise-reducing air passage of a device to provide positive airway pressure in accordance with one embodiment;

FIG. 5 is a three-dimensional schematic diagram of the blower assembly of a device to provide positive airway pressure in accordance with one embodiment;

FIG. 6 is an exploded structural view of the blower assembly of a device to provide positive airway pressure in accordance with one embodiment;

FIG. 7 is a schematic diagram of airflow exiting tangentially to a rotation of the impeller in the blower assembly in accordance with one embodiment;

FIG. 8 is a schematic diagram of the internal gas channel of the blower assembly in accordance with one embodiment;

FIG. 9 is a schematic diagram showing the central axis of the inlet and the central axis of the outlet of a noise-reducing air passage being perpendicular to each other in accordance with one embodiment;

FIG. 10 is a schematic diagram showing the central axis of the intake port and the central axis of the exhaust port of the blower assembly being perpendicular to each other in accordance with one embodiment;

FIG. 11 is a schematic diagram showing the intake port of the blower assembly of a device to provide positive airway pressure provided at an approximately central position of the chamber of the noise-reducing air passage in a top view in accordance with one embodiment;

FIG. 12 is a schematic diagram showing the blower assembly of a device to provide positive airway pressure provided at an approximately central position of the noise-reducing air passage in the vertical direction in accordance with one embodiment;

FIG. 13 is a schematic diagram showing the inlet and outlet of the noise-reducing air passage of a device to provide positive airway pressure not being on the same horizontal plane in accordance with one embodiment;

FIG. 14 is a schematic diagram comparing the area of the outlet of the noise-reducing air passage with the area of the exhaust port of the blower assembly in accordance with one embodiment;

FIG. 15 is a schematic diagram showing the positions of the inlet and outlet of the noise-reducing air passage and the intake port and exhaust port of the blower assembly in accordance with one embodiment;

FIG. 16 is a schematic diagram showing the vertical distance between the noise-reducing air passage and the blower assembly in accordance with one embodiment;

FIG. 17 is a schematic diagram showing the distance between the blower assembly and the inner wall of the noise-reducing air passage in accordance with one embodiment;

FIG. 18 is a schematic diagram comparing the size between the intake port of the blower assembly and the central opening of the impeller in accordance with one embodiment;

FIG. 19 is a schematic diagram showing the axis of the inlet of the noise-reducing air passage being perpendicular to the axis of the intake port of the blower assembly in accordance with one embodiment;

FIG. 20 is a schematic diagram showing the axis of the inlet of the noise-reducing air passage being parallel to the axis of the intake port of the blower assembly in accordance with one embodiment;

FIG. 21 is a schematic diagram showing the bracket for sealing and supporting and transition components of a device to provide positive airway pressure in accordance with one embodiment;

FIG. 22 is a three-dimensional schematic diagram of another form of a device to provide positive airway pressure in accordance with one embodiment;

FIG. 23 is a three-dimensional schematic diagram of the casing with sponge of a device to provide positive airway pressure in accordance with another embodiment.

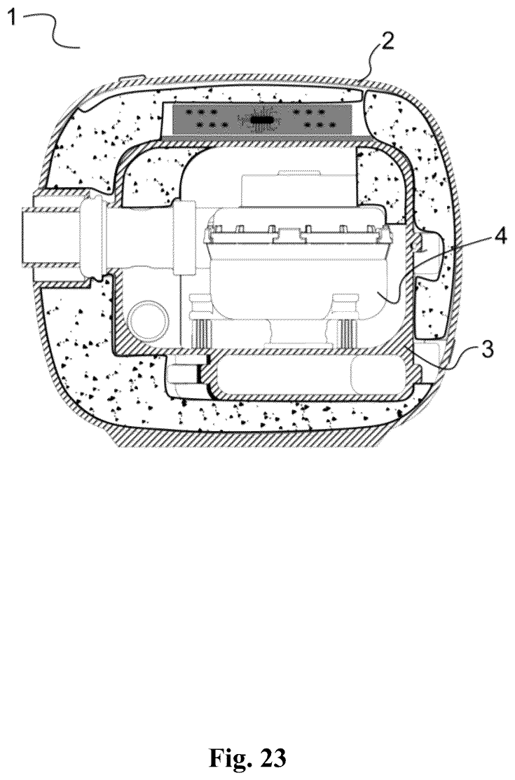

### DETAILED DESCRIPTION

To facilitate the understanding of the disclosure, a more comprehensive description will be provided with reference to the relevant drawings. The drawings illustrate typical embodiments of the disclosure. However, the disclosure can be implemented in many different forms and is not limited to the embodiments described herein. On the contrary, the embodiments are provided to make the disclosure more thorough and comprehensive.

Unless otherwise defined, all technical and scientific terms used herein have the same meaning as commonly understood by one of ordinary skill in the art to which this disclosure belongs. The terms used in the specification of the disclosure herein are for the purpose of describing particular embodiments only rather than limiting the disclosure.

Compared to existing PAP devices on the market, the present disclosure incorporates refined and scientifically tested adjustments to the internal structure and component coordination of the device to provide positive airway pressure. These enhancements improve the advantages of each component, thereby enhancing the overall performance and efficiency of the device. The device provided by this disclosure achieves better noise reduction, efficiency, reliability, and lifespan. Consequently, this device not only enhances performance metrics but also provides patients with a more stable, safe, and comfortable user experience, demonstrating clear competitive advantages in the market. This disclosure benefits patients, manufacturers, and the market by offering a more advanced solution.

Detailed embodiments are presented below to elucidate the configurations of a device to provide positive airway pressure provided by this disclosure, which is used to deliver breathable gas into a patient's airway for treatment of respiratory-related diseases.

### Terminology

Essentially, approximately, substantially and roughly: In certain embodiments of the disclosure, these terms indicate a variation within fifteen percent of the original value.

Air, gas: In some embodiments, “air” and “gas” refer to the breathable air used in daily life. In other embodiments, they refer to other gases or gas mixtures suitable for respiration, such as oxygen-enriched atmospheres.

Environment: In some embodiments, “environment” refers to the area outside the blower assembly within the casing of the noise-reducing air passage. In other embodiments, it refers to the surroundings of the patient's location.

### Embodiment 1

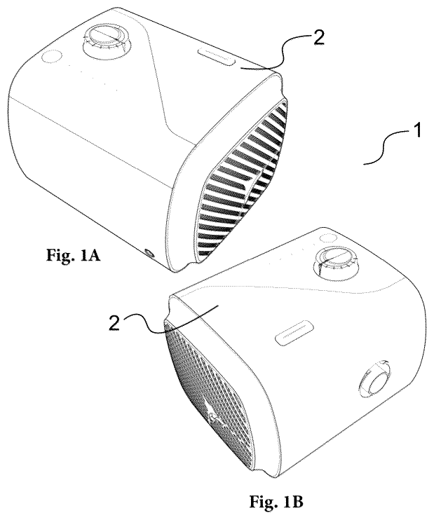

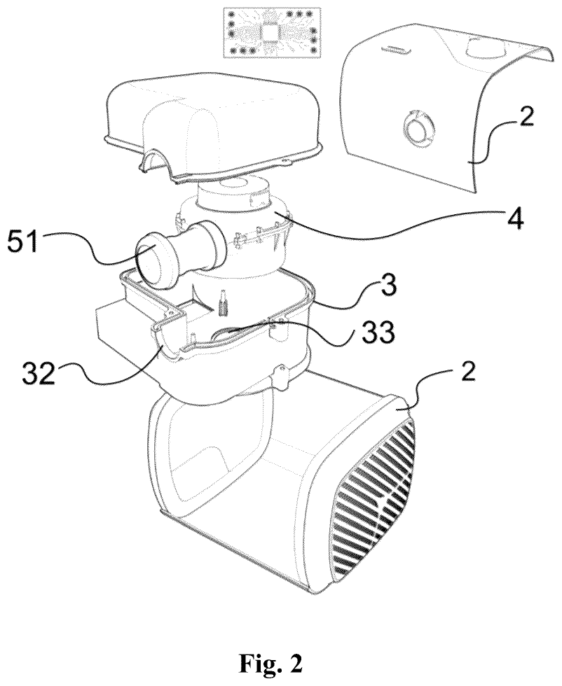

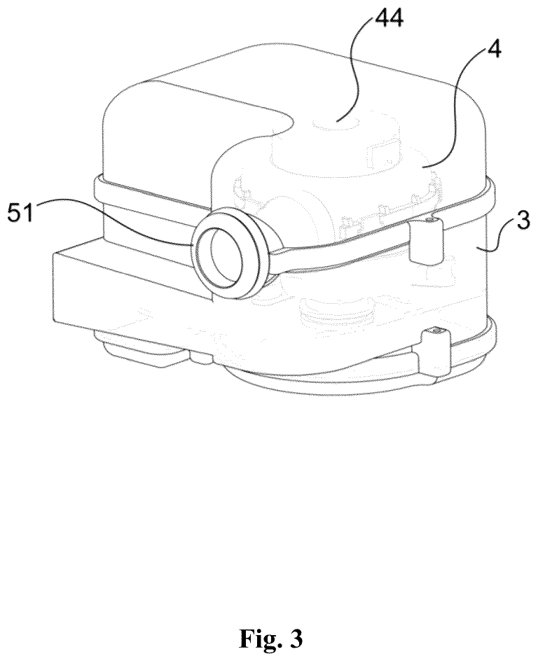

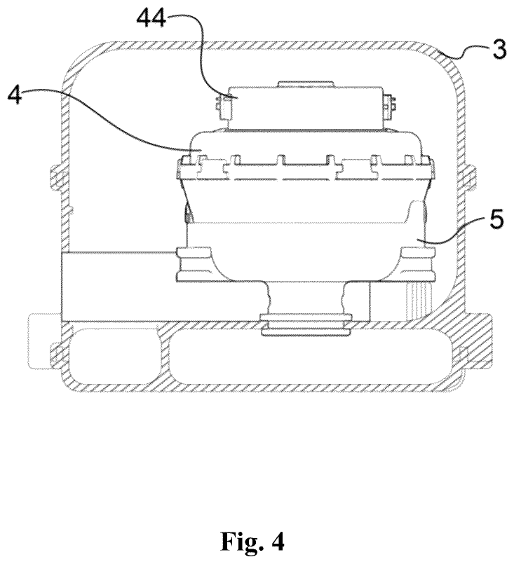

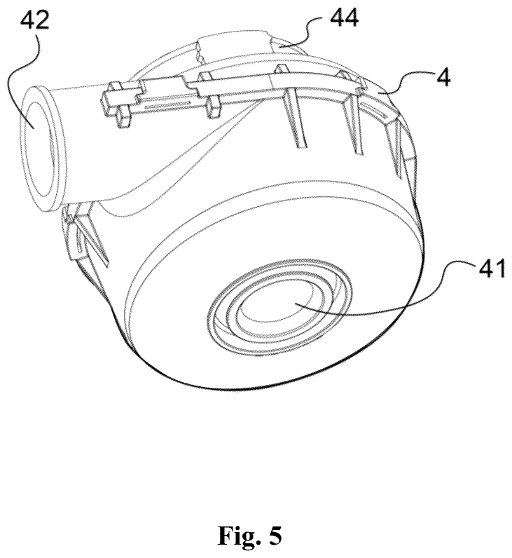

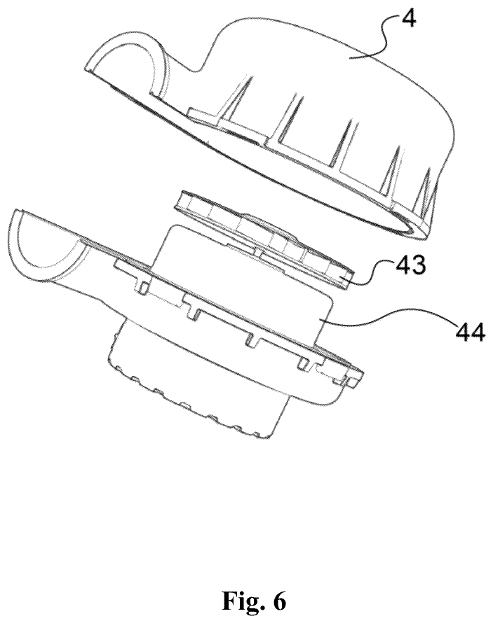

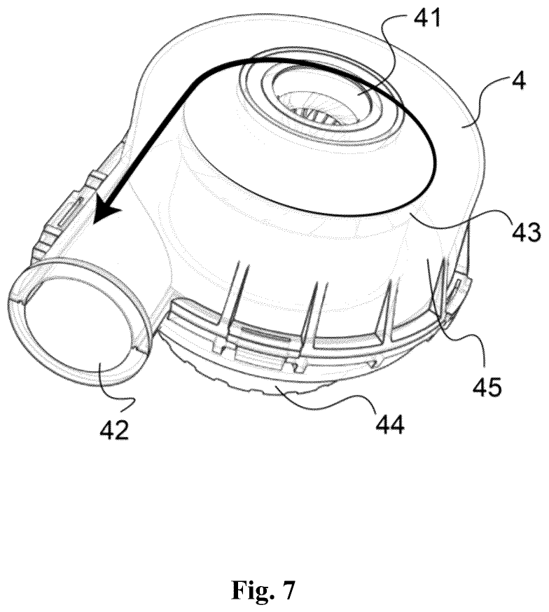

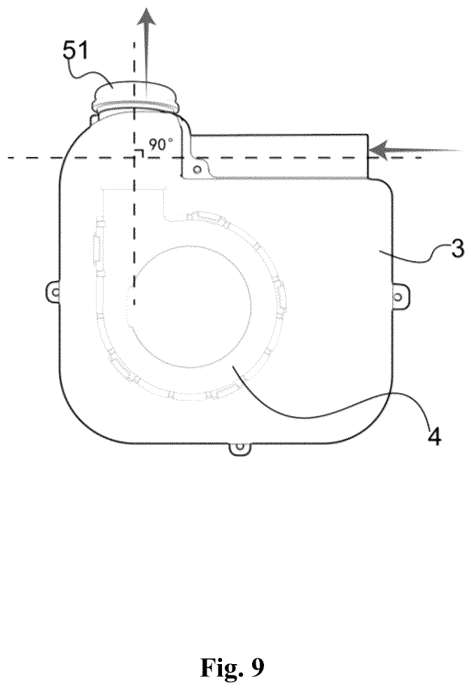

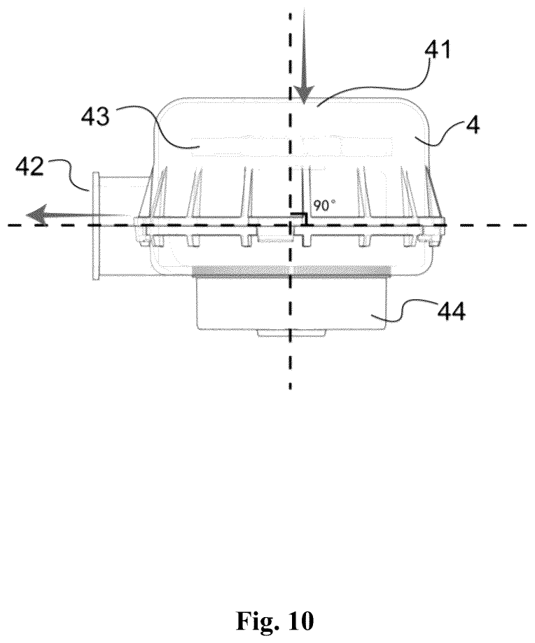

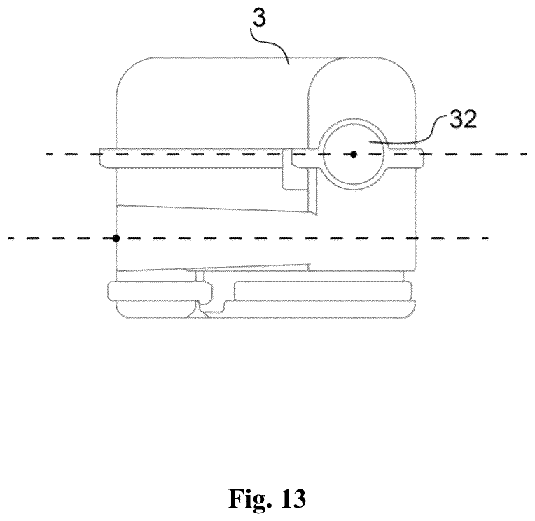

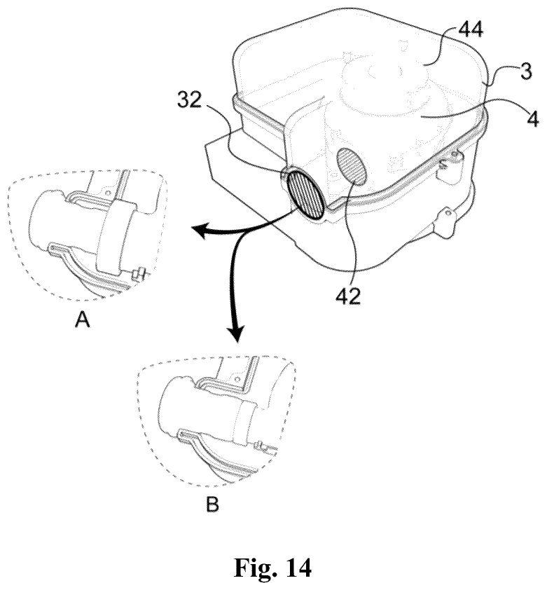

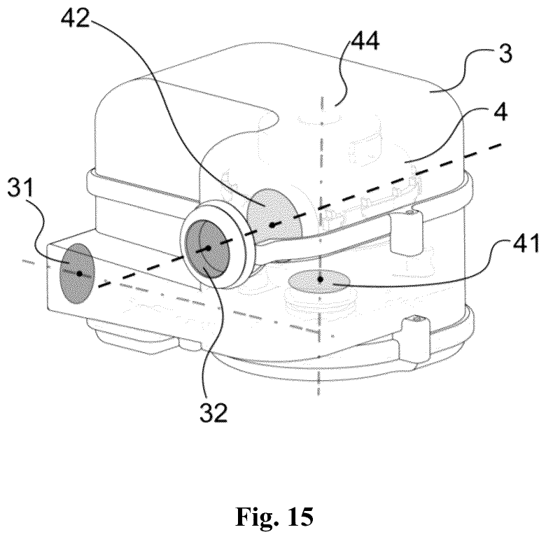

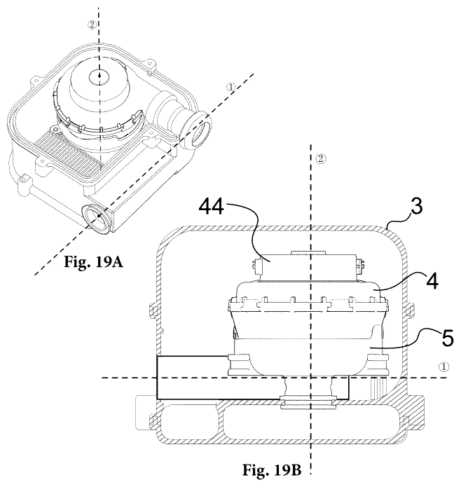

This embodiment provides a device 1 to provide positive airway pressure. This embodiment includes three-dimensional schematic diagrams, exploded structural views, cross-sectional views, airflow path diagrams, component positioning diagrams, and various data diagrams, as referenced in FIGS. 1-19. This embodiment pertains to a device 1 to provide positive airway pressure configured to pressurize breathable gas and deliver it into a patient's airway for the treatment of respiratory-related diseases. The device 1 to provide positive airway pressure includes a casing 2, a noise-reducing air passage 3 and a blower assembly 4. Inside the casing 2, the noise-reducing air passage 3 is provided, which has an inlet 31 and an outlet 32. The blower assembly 4 includes a blower housing, an impeller 43, a motor 44, and an internal gas channel 45. The blower assembly 4 is provided inside the noise-reducing air passage 3 and is configured to pressurize the breathable gas entering the noise-reducing air passage 3 and then output it. The pressurized gas is ultimately conveyed through the outlet 32 of the noise-reducing air passage 3 to the outlet of the casing 2 and then to the patient's airway for therapeutic purposes.

Specifically, the casing 2 is configured to enclose internal components, which include all parts within the casing 2, such as the noise-reducing air passage 3 and the blower assembly 4. In some cases, an outlet pipe is provided at the outlet 32 of the noise-reducing air passage 3. The casing 2 can take various forms, such as spherical, square, roughly conical, or any other shape. Additionally, the casing 2 includes an outlet and an inlet. The outlet of the casing 2 is configured to connect to a breathing hose, typically as an opening, while the inlet of the casing 2 is configured to communicate with the inlet 31 of the noise-reducing air passage 3 inside. This communication means that inlet of the casing 2 is directly or indirectly connected to the inlet 31 of the noise-reducing air passage 3, allowing external air to be drawn in and delivered into the noise-reducing air passage 3 for pressurization by the blower assembly 4. Furthermore, the casing 2 has a robust structural design that effectively protects sensitive electronic components and other critical parts from external impacts. It is made of high-strength, wear-resistant materials, which can be plastic, metal, or other composite materials. In some cases, the casing 2 of the device 1 also features fireproof, dustproof, and heat dissipation properties.

The noise-reducing air passage 3 is configured to pressurize and reduce the noise of the airflow entering it. The device 3 has an inlet 31, an outlet 32, an inner wall, and an outer wall. The inlet 31 of the noise-reducing air passage 3 communicates with the inlet of the casing 2. The inlet 31 of the noise-reducing air passage 3 can be one or multiple openings, with shapes that may be circular, square, oval, or any other form, without affecting the airflow rate. The outlet 32 of the noise-reducing air passage 3 is configured to communicate with the exhaust port 42 of the blower assembly 4, which means that the outlet 32 of the noise-reducing air passage 3 either directly or indirectly connects to the blower assembly 4, delivering the pressurized breathable gas outside the noise-reducing air passage 3. In one configuration, a transition component connects the exhaust port 42 of the blower assembly 4 and the outlet 32 of the noise-reducing air passage 3 to prevent leakage. Typically, the inlet 31 and the outlet 32 are not provided on the same wall of the noise-reducing air passage 3. This separation helps to isolate noise at the inlet 31 and outlet 32 to prevent noise accumulation and consequently reduce overall noise levels. In one implementation, the inlet 31 and outlet 32 of the noise-reducing air passage 3 are not on the same horizontal plane. This arrangement not only separates the inlet 31 and outlet 32 horizontally but also increases their vertical distance, further isolating the noise at the inlet 31 and outlet 32. This configuration also establishes an angle between the central axis of the inlet 31 and the central axis of the outlet 32, which means that the central axis of the inlet 31 and the central axis of the outlet 32 are intersected when the two are projected onto any two-dimensional plane. Additionally, in some cases, an outlet pipe is provided at the outlet 32 of the noise-reducing air passage 3. The outlet pipe includes at least one section of non-tapered cylindrical shape. The presence of the outlet pipe serves two main purposes: firstly, it provides a certain length of wall at the outlet 32, which helps connect the outlet 32 of the noise-reducing air passage 3 to the exhaust port 42 of the blower assembly 4 and guide the airflow exiting from the blower assembly 4. It also assists in connecting the outlet 32 of the noise-reducing air passage 3 to the outlet of the casing 2. Secondly, the outlet pipe with a certain length helps organize and reduce noise from the pressurized airflow delivered by the blower assembly 4, allowing the airflow to stabilize within the pipe. This process aids in reducing turbulence and pressure fluctuations in the airflow, and configuring at least a section of the outlet pipe in a non-tapered cylindrical shape helps prevent abrupt pressure changes that can increase noise. Furthermore, the casing of the noise-reducing air passage 3 has excellent sealing properties, encompassing both the sealing between its own components and the sealing between the casing 2 and the noise-reducing air passage 3. Good sealing performance is crucial to prevent external dust, moisture, or other harmful substances from entering the noise-reducing air passage 3 and affecting airflow. The sealing can be achieved through direct mechanical connections or by using elastomers 51 to form a seal, as well as through adhesives like glue, tape, or other forms of sealing connections. In one implementation, the connection between the casing 2 and the noise-reducing air passage 3 may be airtight, although it can also be non-hermetically sealed. Moreover, the walls of the noise-reducing air passage 3 include one of the following materials: polypropylene, polycarbonate, polyethylene terephthalate-1,4-cyclohexane dimethanol ester, polyamide, or polyetheretherketone.

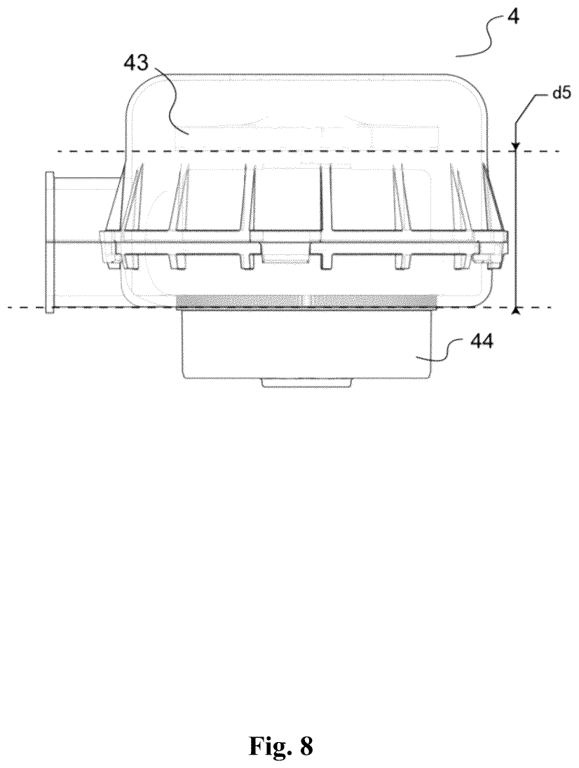

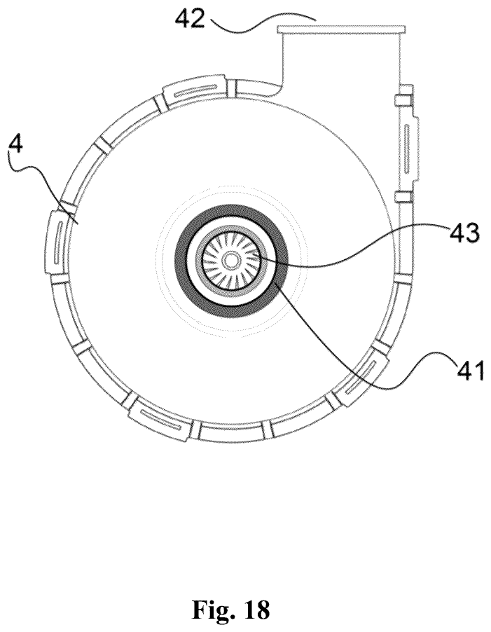

The pressurization function of the noise-reducing air passage 3 is primarily achieved through its internal blower assembly 4, which serves as the core component of the device 1 to provide positive airway pressure. The blower assembly 4 has an intake port 41 and an exhaust port 42, configured to pressurize the gas entering the noise-reducing air passage 3. The central axis of the intake port 41 and the central axis of the exhaust port 42 are perpendicular to each other, allowing the gas to enter through the intake port 41 and flow to the exhaust port tangentially to a rotation of the impeller 43. The blower assembly 4 includes a blower housing, the impeller 43, the motor 44, and its internal gas channel 45, with at least part of the internal gas channel 45 located below the impeller 43. The internal gas channel 45 is positioned to sink to approximately the middle part of the blower assembly 4. The gas channel 45 is set to be submerged to the approximate middle of the blower assembly 4. The ‘approximate middle’ refers to a plane near the furthest point of the impeller 43 within the channel housing the exhaust port 42, designated as the base plane of the gas channel 45. This base plane is provided at least 10 mm above the bottom plane of the impeller 43 near the motor 44 (as shown in FIG. 8, d5≥10 mm). This arrangement enhances the noise reduction and cooling efficiency of the blower assembly 4. The impeller 43 of the blower assembly 4 can take various forms such as forward-curved, backward-curved, closed, or radial impellers. In one implementation, the impeller is a closed impeller, which means it has multiple blades provided between a first and a second thin plate. The first thin plate is provided on the side away from the motor 44. And the impeller 43 has a central opening which extends upward on the side facing the intake port 41 of the blower assembly 4, forming an arc with the bottom plane of the first thin plate. The second thin plate has a central opening in a flat shape. Using this type of impeller 43, in conjunction with a sunken gas channel, enhances the performance of the blower on its existing basis. The motor 44, provided adjacent to the impeller 43, is the core power source of the entire device 1 to provide positive airway pressure, typically employing direct current or alternating current techniques. The motor 44 shows efficient, stable, and reliable performance, providing patients with a continuous and stable power supply. The blower assembly 4 also includes a robust housing that encapsulates the blower, providing sufficient protection for the internal components of the blower assembly 4, and effectively isolating electromagnetic interference and noise from the motor 44. The blower housing generally includes two parts, but the blower can also have a single-piece housing. Near the impeller 43, the housing has an opening forming the intake port 41 of the entire blower assembly 4, and at its edge, it features an exhaust port 42 that may consist of one or two parts. The specific process of pressurizing airflow through the blower assembly 4 involves the airflow first entering the interior of the blower assembly 4 through its intake port 41, immediately being pressurized following the rotation of the impeller 43, then moving into the internal gas channel 45 of the blower assembly 4, and finally exiting from the blower assembly through the exhaust port 42 tangentially to a rotation of the impeller 43. To ensure smooth entry of airflow through the intake port 41 into the blower assembly 4, the intake port 41 should be equal to or larger than the central opening of the impeller 43 (as shown in FIG. 18), allowing the airflow to align with and follow the rotational path of the impeller 43. In one implementation, the air enters the blower assembly 4 through the intake port 41 and flows to the exhaust port 42 tangentially to a rotation of the impeller 43, where the blower assembly provided inside the noise-reducing air passage 3 is an axial-flow blower assembly. From the above discussion, it is evident that the exhaust port 42 of the blower assembly 4 is configured to communicate with the outlet 32 of the noise-reducing air passage 3. To ensure that a sufficient flow of pressurized breathable gas can smoothly exit from the internal gas channel 45 of the blower assembly 4, the area of the outlet 32 of the noise-reducing air passage 3 connected to the exhaust port 42 of the blower assembly 4 is 0.7 to 1.5 times the area of the blower exhaust port 42, with a preferred range of 0.85 to 1.1 times. In one implementation, the exhaust port 42 of the blower assembly 4 and the outlet 32 of the noise-reducing air passage 3 are sealingly connected by an elastomer 51. This configuration ensures that the airflow is sealed during transmission from entering the blower assembly 4 to exiting from the exhaust port 42, reducing the possibility of leakage. In another implementation, there is a transition component between the exhaust port 42 of the blower assembly 4 and the outlet 32 of the noise-reducing air passage 3 that connects the two and prevents air leakage. In one configuration, the intake port 41 of the blower assembly 4 and the inlet 31 of the noise-reducing air passage 3 are non-concentric, whereas the exhaust port 42 of the blower assembly 4 and the outlet 32 of the noise-reducing air passage 3 are concentric.

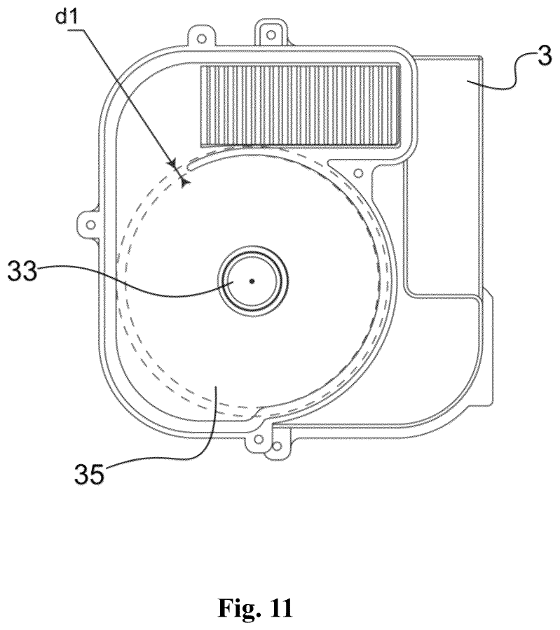

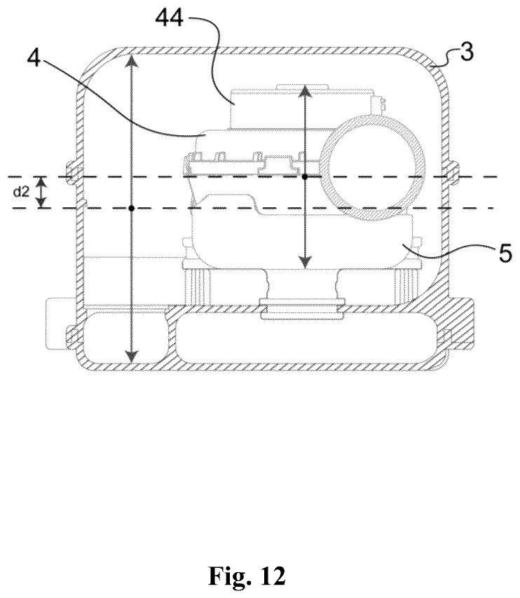

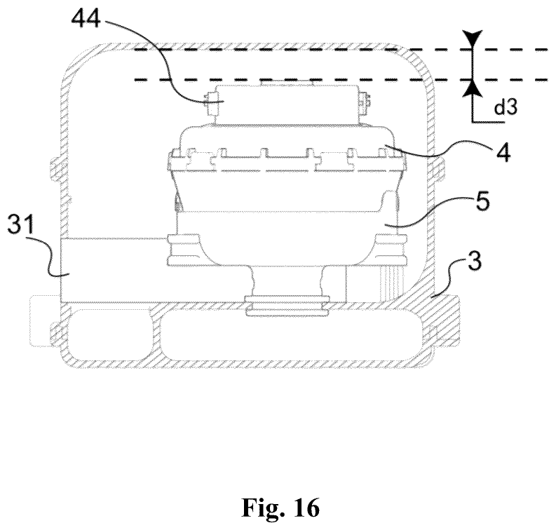

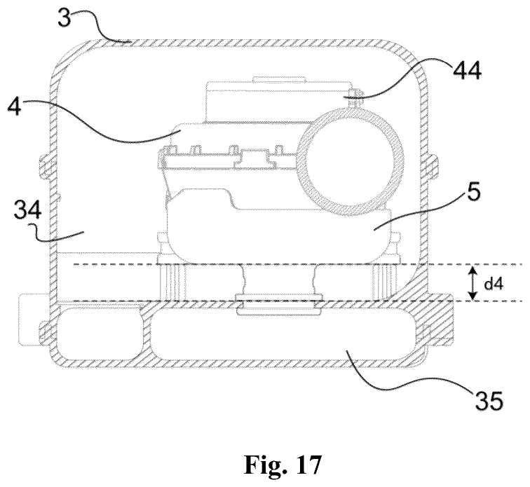

To achieve better noise reduction, this disclosure has made specific requirements regarding the placement and size of the blower assembly 4 within the noise-reducing air passage 3. Theoretically, a larger internal volume of the noise-reducing air passage 3 typically results in lower resistance to gas flow and reduces the likelihood of turbulence. However, excessively large internal volumes can create more dead space, hindering effective gas circulation and thereby increasing noise due to reduced airspeed. Extensive testing and various volume ratio experiments between the noise-reducing air passage 3 and the blower assembly 4 have determined that the volume ratio of the blower assembly 4 to the noise-reducing air passage 3 is between 3 to 18, with a preferred ratio between 4 and 7 for noise reduction. Additionally, for the gas to efficiently and smoothly enter the blower assembly 4, when the blower assembly 4 is secured within the noise-reducing air passage 3, the distance between the blower assembly 4 and the inner wall of the noise-reducing air passage 3 is at least 3 mm (as shown in FIG. 16 and FIG. 17, d3≥3 mm, d4≥3 mm). Moreover, the noise-reducing air passage 3 typically includes two chambers: a first chamber 34 and a second chamber 35 separated by a wall. An opening 33 is provided on the wall and is configured to communicate with the intake port 41 of the blower assembly 4. In this configuration, the blower assembly 4 is secured within one of the two chambers. Specifically, in one case, the blower assembly 4 can be secured in the first chamber 34, and the pressure in the first chamber 34 is higher than that in the second chamber 35. In this embodiment, the blower assembly 4 is inverted and placed within the noise-reducing air passage 3 so that the direction in which breathable air enters through the intake port 41 of the blower assembly 4 is vertical. At this time, the height of the noise-reducing air passage 3 differs from the height of the blower assembly 4 by at least 5 mm. When the orientation of the breathable air entering the blower assembly 4 from the intake port 41 is either vertical or horizontal, the axis of the inlet 31 of the noise-reducing air passage aligns parallel or perpendicular to the axis of the intake port 41 of the blower assembly 4. In one configuration, the blower assembly 4 is provided at an approximately central position of the noise-reducing air passage 3. The central position can be interpreted in two ways: from a top view (where the viewer's line of sight forms a 90-degree angle with the ground or a horizontal plane), the intake port 41 of the blower assembly is provided at an approximately central position of the chamber of the noise-reducing air passage 3. Furthermore, the device 1 to provide positive airway pressure includes a bracket 5, with the blower assembly 4 secured within the noise-reducing air passage 3 by this bracket 5. In one case, the blower assembly 4 is secured to the noise-reducing air passage 3 at an approximately central position in a vertical direction by the bracket 5, where at least part of the bracket 5 is in contact with the wall of the noise-reducing air passage 3 and the contact area between the bracket 5 and the blower assembly 4 is at least 220 mm². This arrangement facilitates a more uniform airflow within the noise-reducing air passage 3 and into the internal gas channel 45 of the blower assembly 4. The bracket 5 has at least two different wall thicknesses, which helps to better reduce noise from the blower assembly 4. When the blower assembly 4 is provided approximately at the center from a top view, the intake port 41 of the blower assembly is provided at an approximately central position of the chamber of the noise-reducing air passage 3. In another embodiment, the intake port 41 of the blower assembly is also approximately centered within the chamber of the noise-reducing air passage 3, but the intake port 41 of the blower assembly 4 and the inlet 31 of the noise-reducing air passage 3 are non-concentric. From the center of the intake port 41 of the blower assembly 4, draw concentric circles until they are tangent to the outer edges of the noise-reducing air passage 3. The radius difference between the tangential circles at the outer edges of the noise-reducing air passage 3 does not exceed 80 mm (as shown in FIG. 11, d1≤80 mm). When the blower assembly 4 is provided within the noise-reducing air passage 3 at an approximately central position in a vertical direction, select a side of the noise-reducing air passage 3, and mark the midpoint of the line connecting the highest and lowest points of the blower assembly 4. Compare this midpoint to the midpoint of the line connecting the highest and lowest points of the entire noise-reducing air passage 3. The distance between these two midpoints should not exceed 80 mm (as shown in FIG. 12, d2≤80 mm).

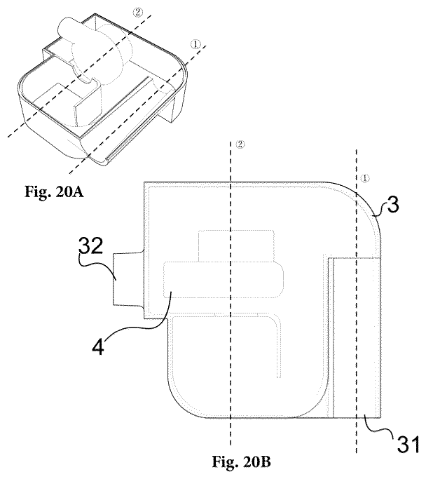

In another embodiment, the blower assembly 4 is provided inside the noise-reducing air passage 3 and the direction of the gas entering the blower assembly 4 through the intake port 41 is horizontal (as shown in FIG. 20).

In another embodiment, the blower assembly 4 is provided at a non-central position within the noise-reducing air passage 3.

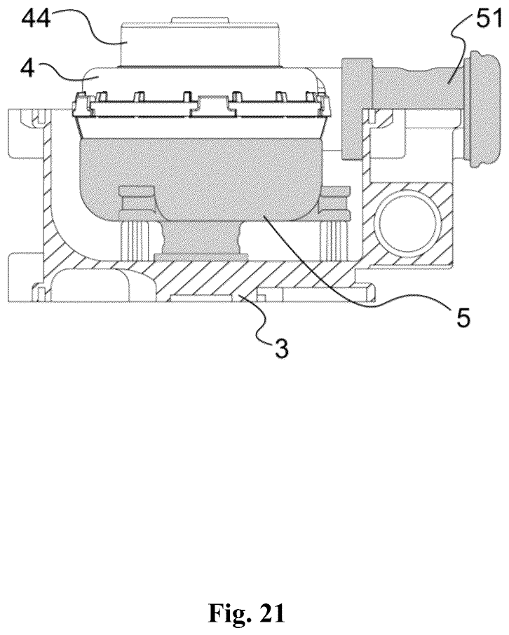

In another embodiment, another form of the bracket 5 within the device 1 to provide positive airway pressure is provided (as shown in FIG. 21).

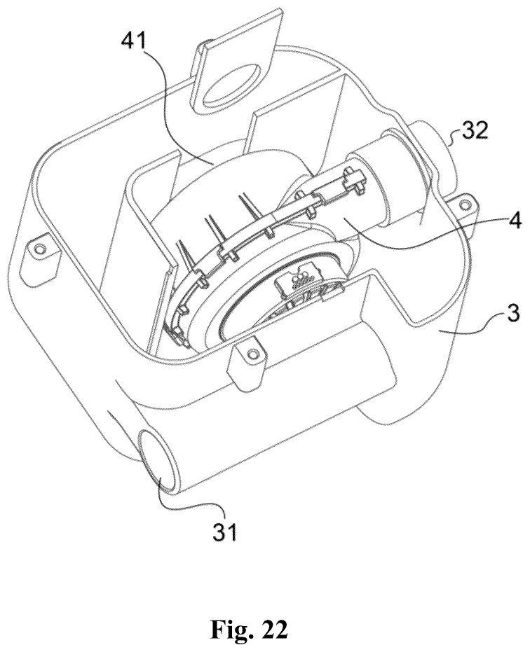

In another embodiment, another form of the noise-reducing air passage 3 within the device 1 to provide positive airway pressure is provided (as shown in FIG. 22).

The implementation of a device to provide positive airway pressure provided by this disclosure at least has the following benefits:
1. By applying scientific theories and experimental data, the internal structure and the placement of components (such as the blower assembly) are optimized to achieve a more efficient and noise-reducing configuration. (1) During the development and design of the internal noise-reducing air passage, the proportional relationship between the blower assembly and the noise-reducing air passage was thoroughly explored to understand its impact on the noise levels of the device. Through extensive data review, designing various proportioned volumes of blower assemblies and noise-reducing air passages, and conducting tests in standard soundproof rooms to ensure accuracy and reliability of the results, it was concluded that a volume ratio of 3 to 18 between the blower assembly and the noise-reducing air passage achieves a more notable noise reduction effect. Utilizing precise theoretical data, the volume ratio between the noise-reducing air passage and the blower assembly was specified more accurately, resulting in better noise reduction performance. Given the increasing demand for quiet performance of the devices to provide positive airway pressure, this disclosure provides an innovative solution to meet this need. By standardizing data, it offers a safe and effective noise reduction method, providing patients with a quieter device, aligning with the growing expectations and requirements for silent therapeutic devices. (2) Positioning the blower assembly centrally within the noise-reducing air passage facilitates a more uniform airflow route and extends the airflow path. In a top view, the blower assembly is provided in a approximately central position within the noise-reducing air passage and the intake port of the blower assembly is also provided at an approximately central position of the noise-reducing air passage. This central placement of the intake port ensures that air can enter the blower assembly evenly from all directions, avoiding localized excessive or insufficient air flows. This configuration reduces the variation in airflow velocity and enhances the stability and uniformity of the airflow. This method also disperses and balances the airflow before it enters the blower assembly, reducing the potential for airflow vibrations and further diminishing turbulence and noise produced by airflow twists and turns. Having the blower assembly provided in a vertically central position within the noise-reducing air passage reduces the distance between the blower assembly and the inner wall of the noise-reducing air passage, which helps to reduce the airflow resistance and pressure fluctuations, thereby enhancing the efficiency and stability of the noise-reducing air passage. The placement of the blower assembly in a vertically central position also extends the airflow path, adding a vertical dimension to the horizontal flow path, which helps the airflow to move more uniformly and stably, thus reducing noise. (3) Setting the outlet and inlet of the noise-reducing air passage on two different planes and with their axes at a perpendicular angle reduces the potential for noise accumulation. When the airflow enters the noise-reducing air passage through the inlet, the characteristics and velocity distribution of the fluid often make the inlet the primary source of airflow noise. Thus, configuring the inlet and outlet perpendicularly helps to separate these two areas, positioning the major noise source away from the patient to achieve noise reduction. (4) Utilizing a quieter centrifugal blower assembly is an effective method to reduce the noise of the device. Since the blower assembly is the principal source of noise in the device, using a quieter blower instead of adding more noise-reduction structures or foam materials fundamentally lowers the noise level of the device. This approach is not only more effective but also more convenient and can enhance the noise performance of the device, improving user comfort and experience quality. In this disclosure, a centrifugal blower assembly is used, which not only produces less noise compared to axial-flow blower assemblies but also offers higher efficiency. Once the blower assembly type is selected, the noise-reducing air passage of this disclosure is designed to complement the chosen blower assembly to achieve the best possible noise reduction in this configuration.
2. Lowering the internal gas channel within the blower assembly presents multiple advantages over existing blower designs in terms of noise reduction and operational efficiency. (1) By relocating the internal gas channel to an approximately central position within the blower assembly, the incoming air passes through the motor, thereby removing heat generated by the motor during operation. This aids in cooling the motor and maintaining it within an optimal temperature range, preventing overheating issues associated with prolonged use. This adjustment not only enhances the motor's efficiency and lifespan but also improves the overall efficiency and durability of the blower assembly. (2) In conventional blower assemblies on the market, the gas channel is designed at the impeller, where the air, immediately after being pressurized by the impeller, enters the gas channel and is expelled. This setup results in sudden and rapid changes in airflow at the impeller and gas channel, which can cause turbulence or irregular flows, thereby increasing the noise level of the blower assembly. Moreover, these sudden changes can induce airflow oscillations, further exacerbating noise issues.

In contrast, sinking the gas channel to a more central position in the blower assembly provides a buffer zone for the pressurized air, allowing the airflow to move more smoothly and uniformly along the path of the gas channel, thereby reducing internal turbulence and irregular flows, and consequently, decreasing noise. (3) When employing a closed impeller in the blower assembly, unlike an open impeller, the blades are enclosed within a sealed casing with a central opening. Air enters through this central opening and moves through the channels formed by the impeller blades, enabling the airflow to exit at a higher pressure from the more enclosed structure. Open impellers, having a more open structural design, allow air to flow freely on both sides of the impeller without much directional constraint. Therefore, using a closed impeller necessitates lowering the gas channel within the blower assembly to provide additional space for integrating and guiding the pressurized airflow, thus enhancing the performance and efficiency of the blower assembly. (4) Designing the gas channel at the central position within the blower assembly also stabilizes the assembly within the noise-reducing air passage. Traditional blower assemblies available on the market often position the internal gas channel at the top of the unit, whereas this disclosure lowers it, effectively shifting the center of gravity downward towards the center of the blower assembly. This design reduces mechanical vibrations caused by changes in airflow pressure and velocity, enhancing the operational stability and safety of the blower. Additionally, it aids the support bracket in securing the blower assembly more stably within the noise-reducing air passage, thereby reducing some motor noise. (5) The disclosure primarily utilizes a centrifugal blower assembly placed inversely within the noise-reducing air passage, altering the direction and speed of air flow. This orientation reduces the formation of vortices and eddies as air flows through the impeller, directed in a specific orientation. Along with the special chamber structure in the disclosure, the noise at the exhaust port of the blower assembly is contained, thereby reducing noise production. In this disclosure, the overall volume of the blower assembly and the volume of the noise-reducing air passage are calculated, and by configuring the gas channel and the components within the chambers (such as the bracket), sound energy is converted into kinetic energy, thereby reducing noise. The noise-reducing air passage is set to enclose the blower assembly at the center and includes chambers at the blower assembly's intake port to use the air or structures within the chambers to lower noise levels. The cooperation between the gas channel and the noise-reducing air passage further adjusts the airflow path to create a vertical disparity, which increases the path of airflow while maintaining the device's flow rate and reduces turbulence at the junctions of various components and chambers, resulting in a smoother airflow path.
3. The bracket system in this disclosure, compared to existing suspensions on the market, not only assists in noise reduction but also provides greater stability for the blower assembly. Part of the bracket is configured to connect to the bottom wall of the chamber housing the blower, with elastomers at the top and around the exhaust port of the blower assembly forming integral parts of the bracket. This design secures the blower assembly at several fixed points, firmly anchoring it within one of the chambers of the noise-reducing air passage. This is more stable than conventional suspension systems and reduces the risk of damage to the blower assembly from vibrations or external impacts. Moreover, the bracket encloses the intake port and exhaust port of the blower assembly, supporting the assembly while also sealing the ports to prevent air leakage.

The above description of the embodiments of the disclosure is provided with reference to the accompanying drawings. However, the disclosure is not limited to the specific embodiments described above. These specific embodiments are merely illustrative and not restrictive. Those skilled in the art, in light of the teachings of the disclosure, may make many modifications and variations without departing from the spirit and scope of the disclosure as defined by the claims. All such modifications and variations are within the protection scope of the disclosure.

It must be noted that as used herein and in the appended claims, the singular forms “a”, “an”, and “the” include their plural equivalents, unless the context clearly dictates otherwise.

1. A positive airway pressure device with a noise-reducing air passage, configured to deliver breathable gas into a patient's airway for treatment of respiratory-related diseases, the device comprising:
a casing, configured to enclose internal components;
the noise-reducing air passage, comprising an inlet, an outlet, an inner surface, and an outer surface, wherein a central axis of the inlet and a central axis of the outlet form an angle;
a blower assembly comprising an intake port and an exhaust port, wherein a central axis of the intake port and a central axis of the exhaust port are perpendicular to each other, wherein the breathable gas is configured to enter the intake port, flow through the blower assembly, and exit the exhaust port tangentially to a rotation of an impeller,
wherein the blower assembly includes a blower housing, the impeller, a motor, and an internal gas channel,
wherein at least part of the internal gas channel of the blower assembly is located on a first side, the first side being the side on which the impeller faces the motor;
wherein a volume ratio of the noise-reducing air passage to the blower assembly is between 3 and 18, and a distance between the blower assembly and the inner surface of the noise-reducing air passage is greater than or equal to 3 mm when the blower assembly is secured within the noise-reducing air passage,
wherein an axis of the inlet of the noise-reducing air passage is in a horizontal direction,
wherein an axis of the outlet of the noise-reducing air passage is in the horizontal direction,
wherein the noise-reducing air passage comprises a first chamber, a second chamber, and a wall to separate the first chamber from the second chamber, the wall having an opening,
wherein the blower assembly is located in the first chamber, and the intake port is in communication with the opening of the wall, and
wherein the wall comprises a vertical portion and a horizontal portion.

2. The device according to claim 1, wherein, in a top view, the intake port of the blower assembly is provided at an approximately central position of the noise-reducing air passage.

3. The device according to claim 1, wherein the noise-reducing air passage includes a first chamber, a second chamber, and a wall to separate the first chamber from the second chamber, and an opening is provided on the wall.

4. The device according to claim 1, wherein the inlet and the outlet of the noise-reducing air passage are not on a same horizontal plane.

5. The device according to claim 1, wherein an area of the outlet of the noise-reducing air passage is 0.7 to 1.5 times an area of the exhaust port of the blower assembly.

6. The device according to claim 1, wherein the device further comprises a bracket and the blower assembly is secured within the noise-reducing air passage by the bracket.

7. A positive airway pressure device with a noise-reducing air passage, configured to deliver breathable gas into a patient's airway for treatment of respiratory-related diseases, the device comprising:
a casing, configured to enclose internal components;
the noise-reducing air passage, comprising an inlet, an outlet, an inner surface, and an outer surface, wherein the noise-reducing air passage is configured to transmit the breathable gas;
a blower assembly comprising an intake port and an exhaust port, wherein the blower assembly is configured to pressurize the breathable gas that enters the noise-reducing air passage,
wherein the blower assembly includes a blower housing, an impeller, a motor, and an internal gas channel, wherein at least part of the internal gas channel is located on a first side, the first side being the side on which the impeller faces the motor;
wherein the device further comprises a bracket, and the blower assembly is secured to the noise-reducing air passage at an approximately central position of the noise-reducing air passage in a vertical direction by the bracket, and wherein the intake port of the blower assembly and the inlet of the noise-reducing air passage are non-concentric, and the exhaust port of the blower assembly and the outlet of the noise-reducing air passage are concentric;
wherein a height of the noise-reducing air passage differs from a height of the blower assembly by at least 5 mm,
wherein an axis of the inlet of the noise-reducing air passage is in a horizontal direction,
wherein an axis of the outlet of the noise-reducing air passage is in the horizontal direction,
wherein, in a top view, the intake port of the blower assembly is provided at an approximately central position of the noise-reducing air passage, and
wherein, in a vertical direction, the blower assembly is located at an approximate central position of the noise-reducing passage.

8. The device according to claim 7, wherein the blower assembly is provided within the noise-reducing air passage and an axis of the intake port of the blower assembly is parallel to a horizontal plane.

9. The device according to claim 7, wherein an outlet pipe is provided at the outlet of the noise-reducing air passage.

10. The device according to claim 7, wherein the exhaust port of the blower assembly is sealably connectable to the outlet of the noise-reducing air passage by an elastomer.

11. The device according to claim 7, wherein the bracket has at least two different wall thicknesses.

12. The device according to claim 11, wherein a contact area between the bracket and the blower assembly is at least 220 mm².

13. The according to claim 11, wherein at least part of the bracket is in contact with the inner surface of the noise-reducing air passage.

14. A positive airway pressure device with a noise-reducing air passage, configured to deliver breathable gas into a patient's airway for treatment of respiratory-related diseases, the device comprising:
a casing, configured to enclose internal components;
the noise-reducing air passage, comprising an inlet, an outlet, an inner surface, and an outer surface, wherein the noise-reducing air passage is configured to transmit the breathable gas;
a blower assembly provided within the noise-reducing air passage, comprising an intake port and an exhaust port, wherein the blower assembly is configured to pressurize the breathable gas that enters the noise-reducing air passage, wherein the exhaust port of the blower assembly is sealably connectable to the outlet of the noise-reducing air passage by an elastomer,
wherein the blower assembly includes a blower housing, an impeller, a motor, and an internal gas channel, wherein at least part of the internal gas channel of the blower assembly is located on a first side, the first side being the side on which the impeller faces the motor;
wherein the impeller is a closed impeller,
wherein an axis of the inlet of the noise-reducing air passage is in a horizontal direction,
wherein an axis of the outlet of the noise-reducing air passage is in the horizontal direction,
wherein, in a top view, the intake port of the blower assembly is provided at an approximately central position of the noise-reducing air passage, and
wherein, in a vertical direction, the blower assembly is located at an approximate central position of the noise-reducing passage.

15. The device according to claim 14, wherein the impeller has a central opening, and the intake port of the blower assembly is greater than or equal to the central opening of the impeller.

16. The device according to claim 14, wherein an axis of the intake port of the blower assembly is perpendicular to a horizontal plane.

17. The device according to claim 14, wherein the inlet and the outlet of the noise-reducing air passage are not on a same horizontal plane.

18. The device according to claim 14, wherein the inner surface and the outer surface of the noise-reducing air passage include one of the following materials: polypropylene, polycarbonate, polyethylene terephthalate-1,4-cyclohexane dimethanol ester, polyamide, or polyetheretherketone.

19. A positive airway pressure device with a noise-reducing air passage, configured to deliver breathable gas into a patient's airway for treatment of respiratory-related diseases, the device comprising:
a casing, configured to enclose internal components;
the noise-reducing air passage, comprising an inlet, an outlet, an inner surface, and an outer surface, wherein the noise-reducing air passage is configured to transmit the breathable gas;
a blower assembly comprising an intake port and an exhaust port, wherein the blower assembly is configured to pressurize the breathable gas that enters the noise-reducing air passage, wherein the blower assembly is secured inside the noise-reducing air passage, wherein the intake port of the blower assembly and the inlet of the noise-reducing air passage are non-concentric, and the exhaust port of the blower assembly and the outlet of the noise-reducing air passage are concentric;
wherein the blower assembly includes a blower housing, an impeller, a motor, and an internal gas channel, wherein at least part of the internal gas channel of the blower assembly is located on a first side, the first side being the side on which the impeller faces the motor;
wherein the impeller is a closed impeller;
wherein the breathable gas is configured to enter the intake port, flow through the blower assembly, and exit the exhaust port tangentially to a rotation of the impeller,
wherein an axis of the inlet of the noise-reducing air passage is in a horizontal direction,
wherein an axis of the outlet of the noise-reducing air passage is in the horizontal direction,
wherein the noise-reducing air passage comprises a first chamber, a second chamber, and a wall to separate the first chamber from the second chamber, the wall having an opening,
wherein the blower assembly is located in the first chamber, and the intake port is in communication with the opening of the wall, and
wherein the wall comprises a vertical portion and a horizontal portion.

20. The device according to claim 19, wherein the noise-reducing air passage includes a first chamber, a second chamber, and a wall to separate the first chamber from the second chamber, and an opening is provided on the wall.

21. The device according to claim 19, wherein the axis of the inlet of the noise-reducing air passage is parallel or perpendicular to an axis of the intake port of the blower assembly.

22. The device according to claim 19, wherein the exhaust port of the blower assembly is sealably connectable to the outlet of the noise-reducing air passage by an elastomer.

23. The device according to claim 19, wherein the impeller has a central opening, and the intake port of the blower assembly is greater than or equal to the central opening of the impeller.
